(19) United States
(12) Patent Application Publication
(10) Pub. No.: US 2020/0051454 A1
(43) Pub. Date: Feb. 13, 2020

(54) DIABETES THERAPY TRAINING DEVICE

(71) Applicant: Becton, Dickinson and Company, Franklin Lakes, NJ (US)

(72) Inventor: Catalina Lopez Giraldo, Medellin (CO)

(73) Assignee: Becton, Dickinson and Company, Franklin Lakes, NJ (US)

(21) Appl. No.: 16/498,603

(22) PCT Filed: Apr. 19, 2018
(86) PCT No.: PCT/US18/28270

§ 371 Date: Sep. 27, 2019

Related U.S. Application Data
(60) Provisional application No. 62/488,003, filed on Apr. 20, 2017.

(51) Int. Cl. G09B 19/00; G09B 5/02; G09B 23/28; G16H 20/17; G16H 20/30; G16H 20/60

(52) U.S. Cl. CPC G09B 19/00; G09B 5/02; G16H 20/60; G16H 20/17; G16H 20/30; G09B 23/285

(57) ABSTRACT

A portable device (20) for educating a user in diabetes management includes a display (26) presenting a user interface to provide a virtual patient (28) having diabetes and depicted as an avatar having the form of a boy or girl. A health status (30) is indicated by a bar graph (32). The user interface presents a feeding control (40) for providing food or drinks to the virtual patient; an exercise control (38) for causing the virtual patent to exercise; and a medication control for providing medication to the virtual patient. Each of the controls causing a corresponding change in the health status of the virtual patient having type-1 diabetes. An injection training application (34) is provided for teaching injection techniques. A testing training application (36) allows the user to check one or more health parameters of the virtual patient. Other applications, such as exercise and diet logging applications and an activities checklist are included as well.

### CROSS REFERENCE TO RELATED APPLICATION

This is a US National Stage entry of PCT application number PCT/US2018/028270, filed Apr. 19, 2018, which claims the benefit of U.S. provisional patent application No. 62/488,003 filed Apr. 20, 2017 the contents of which are incorporated herein by reference in its entirety.

### FIELD OF THE INVENTION

The present invention is directed to a device for assisting children to learn and comply with positive behaviors related to diabetes care.

### BACKGROUND

Living with diabetes is a big responsibility. Patients are required to learn and maintain good habits for food, exercise, medicine doses and times, and injection techniques. There is a particular challenge with children 3 to 10 years old. If diabetes is not controlled with good behavioral habits, risks include serious health complications and even death. 542,000 children are living with Type 1 diabetes, and each year 86,000 more children develop Type 1 diabetes. These children require insulin therapy.

### SUMMARY

In order to better serve children with diabetes, a portable device is provided to train children positive behaviors for coping with diabetes. The device provides an interactive experience, and is an interactive game geared towards children 3 to 10 years old. The portable device, and the software running on the device teaches children to be responsible for an avatar, pet, or the like, represented on the device, and whom the device simulates having diabetes. Accordingly, children are encouraged to care for the avatar with proper food, exercise, medication, and the like. In a fun way, with feedback, children are able to learn how to manage Type 1 diabetes.

### BRIEF DESCRIPTION OF THE DRAWINGS

Further details, features and advantages of designs of the invention result from the following description of embodiment examples in reference to the associated drawings.

FIG. 1 is a front view of a portable device presenting a user interface according to an exemplary embodiment of the invention;

FIG. 2 is a front view of two portable devices, each presenting a user interface showing an avatar in one of two different states according to an exemplary embodiment of the invention;

FIG. 3 is a front view of two portable devices, each presenting a user interface showing a different avatar in one of two different states according to an exemplary embodiment of the invention;

FIG. 4 is a front view of a portable device presenting a user interface with an index of four different icons, and with a listing of other icons according to an exemplary embodiment of the invention;

FIG. 5 is an example user interface for an injection training application according to an exemplary embodiment of the invention;

FIG. 6 is an example user interface for a diet logging application according to an exemplary embodiment of the invention; and

FIG. 7 is an example membership card for use with the portable device according to an exemplary embodiment of the invention.

Throughout the drawings, like reference numbers should be understood to represent like elements, features and structures.

### DETAILED DESCRIPTION

Recurring features are marked with identical reference numerals in the figures in which a portable device 20 for educating a user in diabetes management is disclosed. The portable device 20 is targeted for a user who is a child who has type-1 diabetes or who has a close relationship with another person having diabetes.

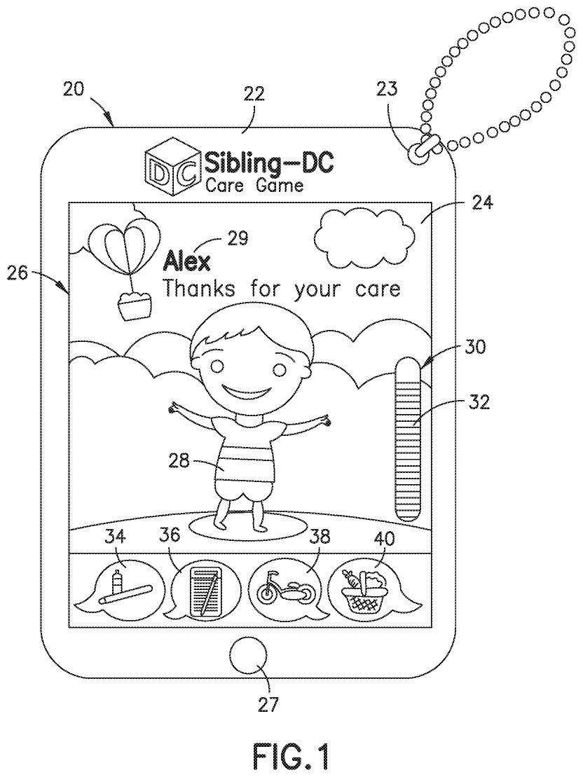

FIG. 1 illustrates an exemplary embodiment of the invention, which is a portable device 20 including a housing 22 defining a hole 23 for receiving a tether, which may be a ball chain as shown in FIG. 1. The hole 23 may also allow the portable device 20 to be connected to a lanyard or a keychain, or other object, which may help to prevent it from being lost or damaged. The housing 22 holds a display 24 such as a LCD or OLED screen. The display 24 may be an e-ink display. The display may be backlight for enhanced visibility in low-light areas. The display 24 presents a user interface 26, which is preferably a graphical user interface (GUI). The housing 22 also includes a home button 27 located below the display 24 for causing the user interface 26 to show a predetermined “home” screen or default interface.

The portable device 20 includes a processor (not shown) such as, for example, a microprocessor, microcontroller, or system-on-chip (SoC), and a machine-readable storage memory holding software instructions for execution by the processor to provide a virtual patient 28 having diabetes. The virtual patient 28 is depicted in the FIGS. as an avatar having the form of a boy or girl. The avatar may have other forms such as, for example, an animal, or a cartoon character. The virtual patient 28 may also be depicted symbolically, for example, by one or more graphs or other depictions of various characteristics. The user may chose the type of avatar, which may include the gender of the virtual patient 28. The user may also provide a name 29 which is shown on the user interface 26.

As also shown on FIG. 1, a health status 30 is indicated on the user interface 26 by a bar graph 32. The health status 30 may be indicated in other ways such as textually, graphically, and/or symbolically. The health status 30 may represent a general health level of the virtual patient 28. Alternatively, the health status 30 may be represented as one or more specific parameters related to the treatment of diabetes in the virtual patient 28. Such specific parameters may include, for example, a blood sugar level, a ketone level, hunger level, hydration level, and fatigue level.

The software preferably provides a fun and portable operating system that transforms children's anguish about not understanding the changes in their body that will teach the children with an impactful challenge that will generate positive change in their routine of care. The software is programmed to interact with the child through the user interface 26, and to contain information required for a diabetic pediatric patient. The child is responsible for the care of the virtual patient 28, including a schedule for food, medicines, activities, physician control, and injection techniques.

Practice with the portable device 20 increases knowledge and awareness for the child as a diabetic patient, as they learn provide the best care for their virtual patient 28. The software preferably teaches metabolic control, adherence tolerance, and correct injection technique, among other things. Preferably the software provides increasing challenges as the child improves, and motivates the child to continue learning and improving.

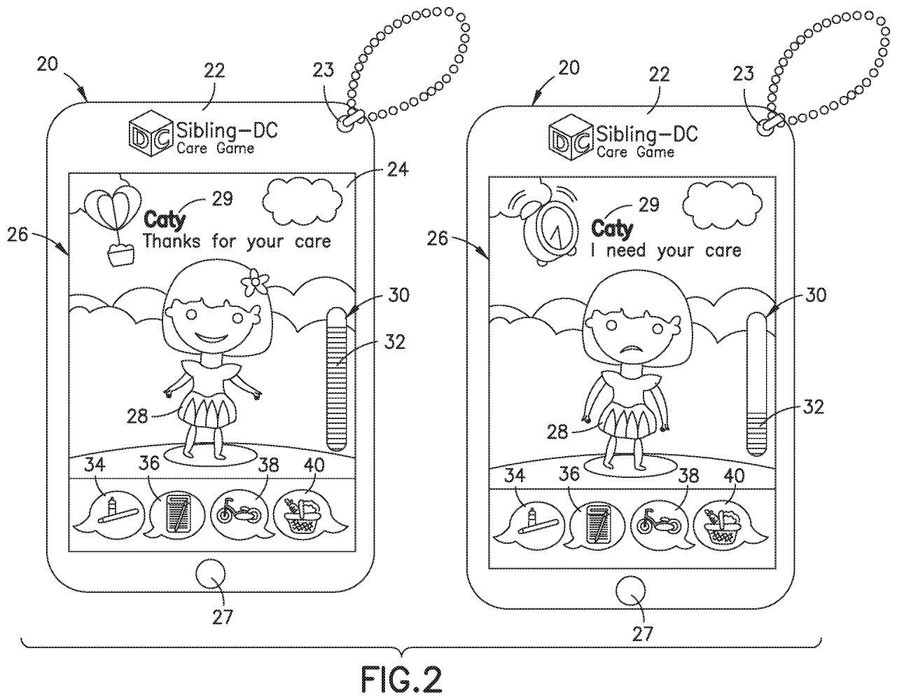

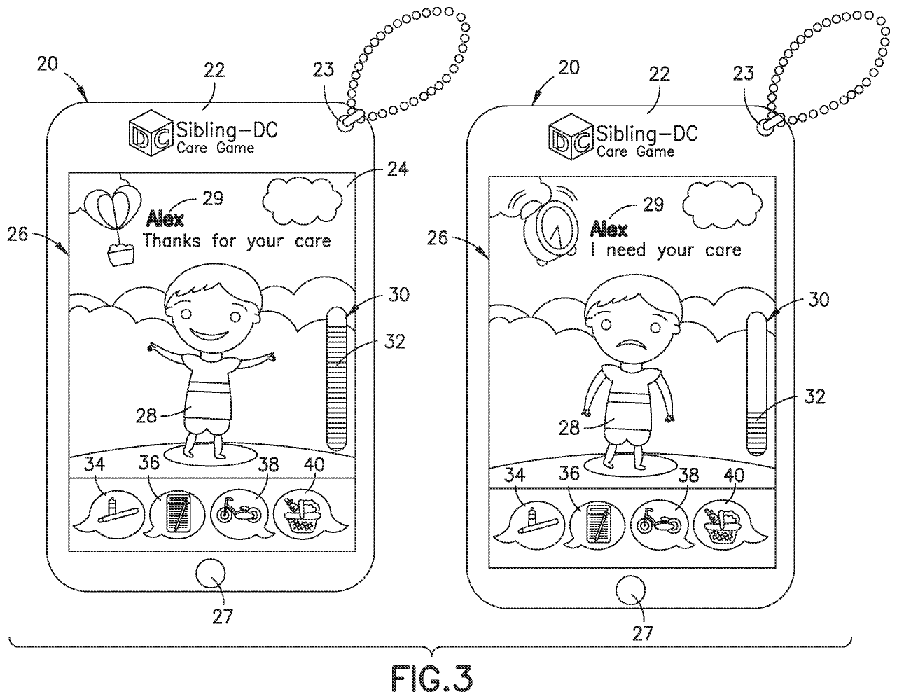

FIG. 2 illustrates an example of a virtual patient 28 represented by a girl avatar having a low health status 30 level and in need of care on the right-side one of the portable devices 20, and with a higher health status 30 level after having received care from a user on the left-side one of the of the portable devices 20. Similarly, FIG. 3 is an example of a virtual patient 28 represented by a boy avatar having a low health status 30 level and in need of care on the right-side one of the portable devices 20, and with a higher health status 30 level after having received care from a user on the left-side one of the of the portable devices 20.

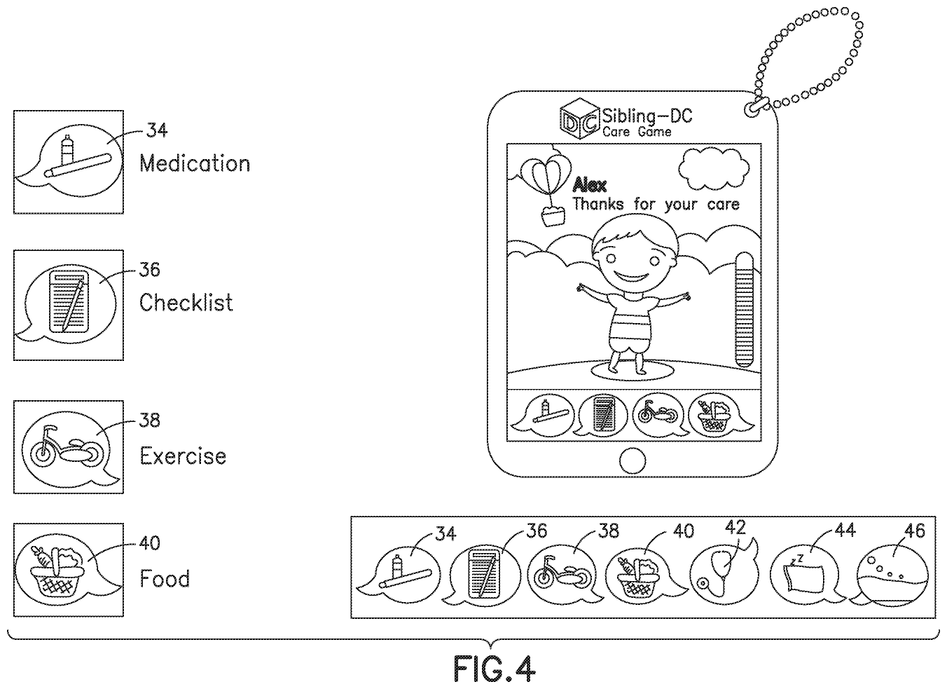

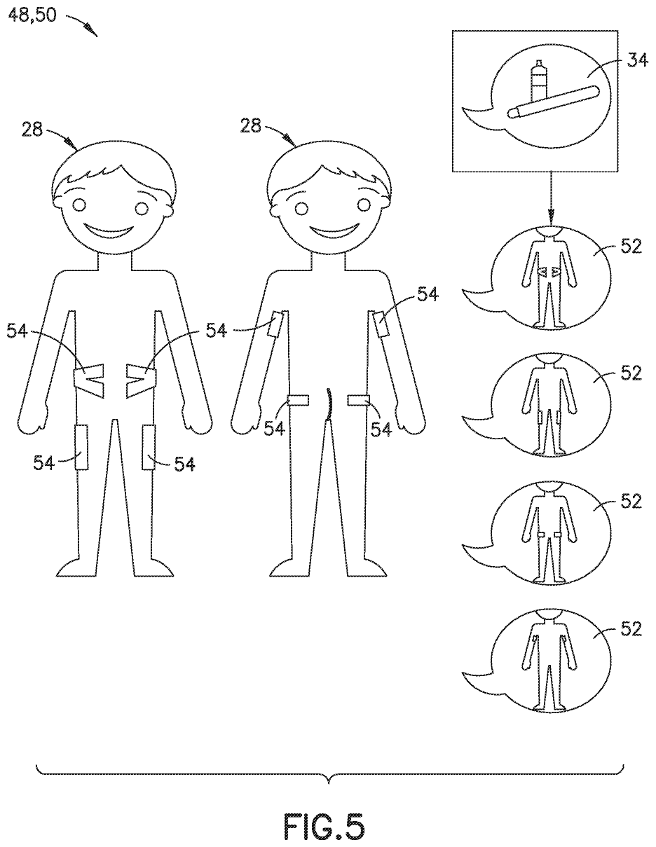

As shown in FIG. 4, the user interface 26 presents a plurality of icons 34, 36, 38, 40, 42, 44, 46. The icons 34, 36, 38, 40, 42, 44, 46 include a medication icon 34 invoking an injection training application 42, shown in FIG. 5, for providing medication to the virtual patient 28, and a checklist icon 36 invoking an activities checklist, allowing the user to verify that different activities related to the management of diabetes are being performed. The activities listed on the activities checklist may include, for example, exercising, resting, feeding, administration of medication, and testing the patient's blood sugar.

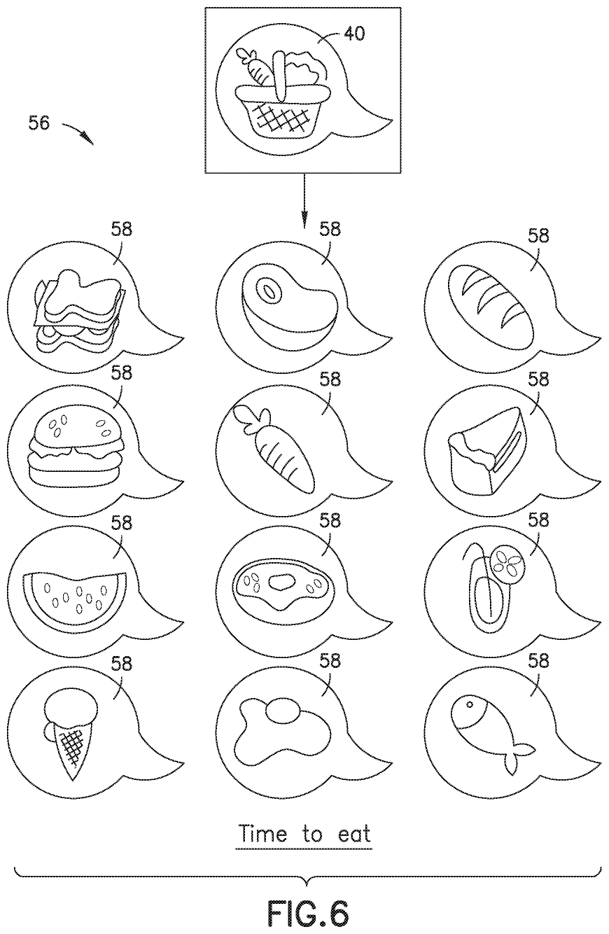

The icons 34, 36, 38, 40, 42, 44, 46 also include an exercise icon 38 invoking an exercise control for the user to direct virtual patent 28 to exercise. The exercise icon 38 may additionally or alternatively invoke an exercise logging application for keeping track of exercise activities done by the virtual patient 28. The icons 34, 36, 38, 40, 42, 44, 46 also include a food icon 40 invoking a feeding control 56, shown in FIG. 6, for providing food or drinks to the virtual patient 28. The food icon 40 may additionally or alternatively invoke a diet logging application for keeping track of food and drinks consumed by the virtual patient 28. As also shown in FIG. 4, the user interface 26 presents other icons 42, 44, 46 representing other controls or settings including a doctor checkup icon 42, a resting or sleeping icon 44, and a bathing icon 46 for the user to direct the virtual patient 28 to perform related actions.

Each of the exercise control, the feeding control 56, and the injection training application 48 causes a corresponding change in the health status 30 of the virtual patient 28 having type-1 diabetes. Each of the exercise control, the feeding control 56, and the injection training application 48 may cause changes to other parameters of the virtual patient 28 having type-1 diabetes, such as, for example, hunger, tiredness level, blood sugar level, etc. Those other parameters may be directly or indirectly presented on the user interface 26.

FIG. 5 illustrates an injection training interface 50 of the injection training application 48 for teaching injection techniques. The injection training interface 50 may also teach about various aspects of the patient's body and physiology as it is effected by diabetes. The injection training interface 50 includes a plurality of injection controls 52 for a user to administer medication to associated injection sites 54 on a body of the virtual patient 28. In this way, the user can learn about specific injection sites 54 and about techniques for administering injected medication to those injection sites 54 such as, for example, rotating between different ones of the injection sites 54.

FIG. 6 illustrates an interface for the feeding control 56, allowing the user select food and drink items 58 for consumption by the virtual patient 28. The feeding control 56, which may also function as a diet logging portion of the software, therefore teaches the user about what and when to eat while managing diabetes.

According to a further aspect, the user interface 26 may include a testing training application (not shown in the figures) for checking a health parameter of the virtual patient. The testing training application may allow, for example, the user to test a blood sugar level or a ketone level of the virtual patient. The testing training application may allow the user to become familiar with the techniques and results of testing procedures used in the management of diabetes.

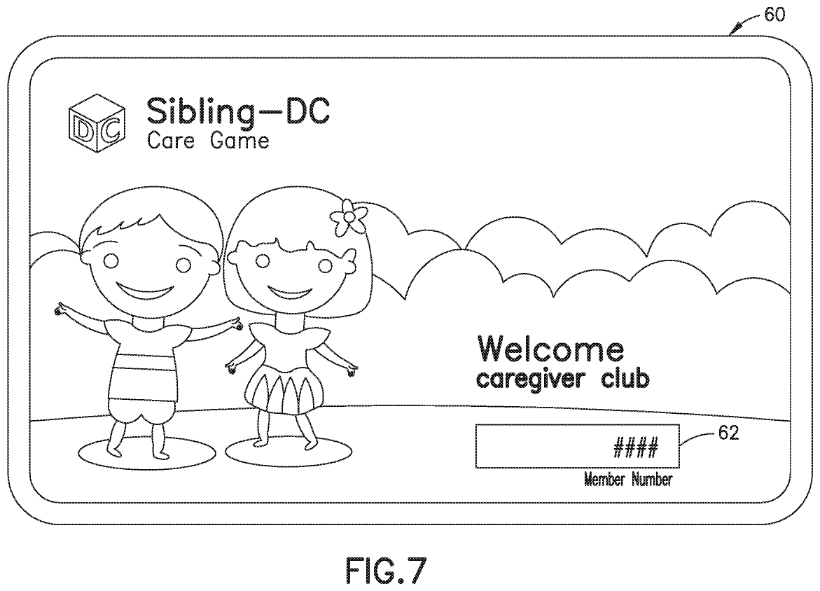

FIG. 7 illustrates an exemplary membership card, representing a reward 60, or the like, provided by the software as an incentive for successful use of the device. As shown, the membership card includes a member number 62 which may uniquely identify the user and/or the virtual patient 28.

The system, methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or alternatively, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices as well as heterogeneous combinations of processors processor architectures, or combinations of different hardware and software, or any other machine capable of executing program instructions.

Thus, in one aspect, each method described above and combinations thereof may be embodied in computer executable code that, when executing on one or more computing devices performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

Obviously, many modifications and variations of the present invention are possible in light of the above teachings and may be practiced otherwise than as specifically described while within the scope of the appended claims.

What is claimed is:

1. A portable device for educating a user in diabetes management, comprising:
a housing having a display presenting a user interface;
software running on the device providing a virtual patient having diabetes on the display and having a health status indicated on the user interface;
a feeding control on the user interface for providing food to the virtual patient;
an exercise control on the user interface for causing the virtual patent to exercise; and
a medication control on the user interface for providing medication to the virtual patient.

2. The portable device for educating a user in diabetes management as set forth in claim 1, wherein said software running on the device presents an avatar graphically representing the virtual patient on the user interface.

3. The portable device for educating a user in diabetes management as set forth in claim 1, wherein said software running on the device further includes an injection training application to teach injection techniques.

4. The portable device for educating a user in diabetes management as set forth in claim 3, wherein said injection training application includes injection controls for administering an injection to injection sites on a body of the virtual patient.

5. The portable device for educating a user in diabetes management as set forth in claim 1, wherein said software running on the device further includes an activities checklist.

6. The portable device for educating a user in diabetes management as set forth in claim 1, wherein said software running on the device further includes an exercise logging application.

7. The portable device for educating a user in diabetes management as set forth in claim 1, wherein said software running on the device further includes a diet logging application.

8. The portable device for educating a user in diabetes management as set forth in claim 1, wherein said health status of the virtual patent is represented by a bar graph on said user interface.

9. The portable device for educating a user in diabetes management as set forth in claim 1, wherein said health status of the virtual patent is represented by a facial expression or body language of the virtual patient on said user interface.

10. The portable device for educating a user in diabetes management as set forth in claim 1, wherein said health status of the virtual patent is a general health level.

11. The portable device for educating a user in diabetes management as set forth in claim 1, wherein said health status of the virtual patent is a blood sugar level.

12. The portable device for educating a user in diabetes management as set forth in claim 1, wherein said software running on the device further includes a testing training application for checking a health parameter of the virtual patient.

13. The portable device for educating a user in diabetes management as set forth in claim 12, wherein said health parameter of the virtual patient includes a blood sugar level.

14. The portable device for educating a user in diabetes management as set forth in claim 12, wherein said health parameter of the virtual patient includes a ketone level.

15. The portable device for educating a user in diabetes management as set forth in claim 1, wherein said software provides the user with a reward as an incentive for successful use of the device.
